(19) United States
(12) Plant Patent Application Publication
(10) Pub. No.: US 2017/0374792 P1
(43) Pub. Date: Dec. 28, 2017

(54) Portulaca plant named 'PHSFR11-0'

(50) ;

(71) Applicant: Steve Jones, Santa Monica, CA (US)

(72) Inventor: Steve Jones, Santa Monica, CA (US)

(21) Appl. No.: 14/999,760

(22) Filed: Jun. 22, 2016

(51) Int. Cl. A01H 5/02

(52) U.S. Cl. CPC A01H 5/02
USPC PLT/471

(57) ABSTRACT

A new and distinct Portulaca cultivar named ‘PHSFR11-0’ is disclosed, characterized by unique bi-color or tri-color foliage and low, densely growing, spreading plants. The new variety is a Portulaca, normally produced as an outdoor garden or container plant.

Latin name of the genus and species: Portulaca oleraea.

Variety denomination: ‘PHSFR11-0’.

### BACKGROUND OF THE INVENTION

The new Portulaca cultivar is a product of chance discovery by the inventor at a commercial greenhouse in Santa Paula, Calif. The breeder discovered an interesting, naturally occurring branch mutation in a planting of unnamed, unpatented Portulaca oleraea.

The branch mutation showed bi-color foliage on a plant which was otherwise entirely green in foliage color. This discovery was made during May of 2015.

Asexual reproduction of the new cultivar was first performed by vegetative terminal cuttings. at the same commercial greenhouse in Santa Paula, Calif. in August of 2015. Subsequent propagation and growing the plants over a period of several months shows the variegation pattern is expressed as both bi-color and tricolor. The variegation pattern has shown consistency and stability over the course of three propagative generations.

### SUMMARY OF THE INVENTION

The cultivar ‘PHSFR11-0’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘PHSFR11-0’ These characteristics in combination distinguish ‘PHSFR11-0’ as a new and distinct Portulaca cultivar:

1. Unique variegated foliage which can by bi-color or tri-color.
2. Low spreading and dense plant habit.
3. Bright pink flowers.

### COMMERCIAL COMPARISON

To the best of the inventor's knowledge, the new variety is the only Portulaca oleracea plant with variegation and a dense, low growing plant form. Plants of the new cultivar ‘PHSFR11-0’ can however be compared to the unpatented commercial Portulaca ‘Cupcakes Grape Jelly’. The two Portulaca varieties are similar in most horticultural characteristics; however, the new variety differs in the following:

1. Leaves or the new variety are variegated, foliage of the comparator is solid green.
2. Stems of the new variety are flushed pink, stems of the comparator are green.
3. Plant habit of the new variety is low growing a spreading, while the plant habit of ‘Cupcakes Grape Jelly’ is semi-upright.

Plants of the new cultivar ‘PHSFR11-0’ can also be comparable to the unpatented commercial variety can also be comparable to the patented commercial variety Portulaca ‘SAKPOR001’ U.S. Plant Pat. No. 24,528. The two Portulaca varieties are similar in most horticultural characteristics; however, the new variety differs in the following:

1. Leaves or the new variety are variegated, foliage of the comparator is solid green.
2. Stems of the new variety are flushed pink, stems of the comparator are green.
3. Plant habit of the new variety is low growing a spreading, while the plant habit of the comparator is semi-upright and semi-mounding.
3. Plant vigor of the new variety is moderate; this comparator has a vigorous growth habit.

### BRIEF DESCRIPTION OF THE PHOTOGRAPHS

The accompanying photograph in FIG. 1 illustrates in full color a typical plant of ‘PHSFR11-0’ grown in a greenhouse, in Santa Paula, Calif. in a 10 inch basket. Age of the plant photographed is approximately 20 weeks from a rooted cutting.

FIG. 2 illustrates a close up of the foliage. The photographs were taken using conventional techniques and although colors may appear different from actual colors due to light reflectance it is as accurate as possible by conventional photographic techniques.

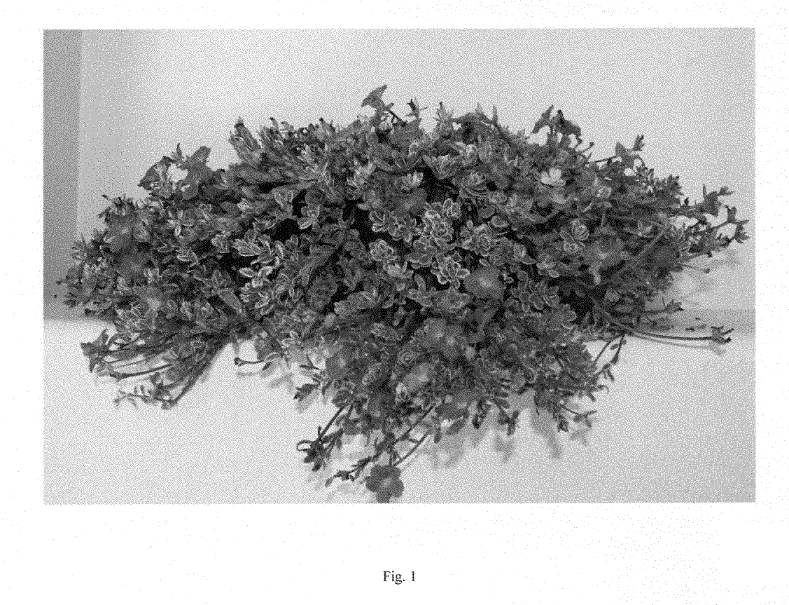

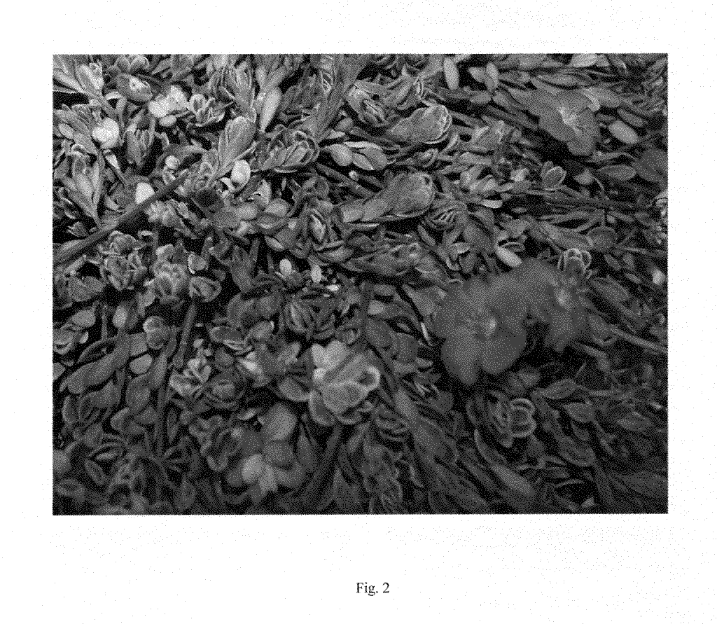

### DETAILED BOTANICAL DESCRIPTION

In the following description, color references are made to The Royal Horticultural Society Colour Chart 2007 except where general terms of ordinary dictionary significance are used. The following observations and measurements describe ‘PHSFR11-0’ plants grown from approximately mid December until mid June in a greenhouse, in Santa Paula, Calif. The growing temperature ranged from 20° C. to 30° C. during the day and from 15° C. to 25° C. during the night. General light conditions are bright, normal sunlight. Measurements and numerical values represent averages of typical plant types.
Botanical classification: Portulaca ‘PHSFR11-0’.
Propagation:
   Time to initiate roots.—About 20 days at approximately 20 to 22° C.
   Time to initiate roots.—10-14 days at approximately 18-21° C.
   Root description.—Fibrous, spreading and dense.
Plant:
   Growth habit.—Low and spreading, semi-trailing and dense.
   Pot size of plant described.—6 inch.
   Height.—Approximately 5 cm
   Plant spread.—Approximately 30 cm.
   Growth rate.—Slow to moderate.
   Branching characteristics.—Very free-branching.
   Branching characteristics.—Tight, compact, dense branching.
   Length of primary lateral branches.—12 cm.
   Diameter of lateral branches.—0.3-0.4 cm.
   Quantity of primary lateral branches.—About 25.
   Characteristics of primary lateral branches.—Form: Round with approximately 4 to 6 ridges. Diameter: Approximately 0.4 cm. Color: Near RHS Greyed-Red 182B and Greyed-Purple 186B. Both colors present in flushes. Closest to the stem apex, RHS Yellow-green 145C. Texture: Glabrous Strength: Flexible
   Internode length.—Average range 1.0 to 1.5 cm.
Foliage:
   Leaf.—Arrangement: Alternate Quantity: Approximately 16 to 22 per branch. Average Length: Average range from 1.8 to 2.8 cm. Average Width: Average range from 1.0 to 1.7 cm. 14. Shape of blade: Oblanceolate to nearly spatulate as foliage ages Apex: Rounded. Base: Acuminate Margin: Entire Texture of top surface: Smooth Texture of bottom surface: Smooth Pubescence: Absent Aspect: Flat, angle of attachment about 75° Color: Young foliage upper side: RHS Green 137C, margin Yellow 2C. Occasional marginal flush near Red 54C, and fine marginal edge near Red-Purple 61B and N66A. Young foliage under side: RHS Green 138B, margin Yellow 5D. Mature foliage upper side: RHS Green 137B, margin Yellow 8C and 8D. Marginal flush near Red 54C, and fine marginal edge near Red-Purple 61B and N66A. Mature foliage under side: RHS Green 138B, margin Yellow 8C and 8D. Marginal flush near Red-Purple N66C. Fine marginal edge near Red-Purple N66A. Venation: Indistinguishable from leaf blade. Petiole: Absent.
Flower:
   Natural flowering season.—Spring to Autumn in Santa Paula, Calif.
   Days to flowering from rooted cutting.—About 6 to 8 weeks
   Inflorescence and flower type and habit.—Terminal inflorescence. Single rotate flower.
   Rate of flower opening.—3 to 5 days from bud to fully opened flower.
   Flower longevity on plant.—Approximately 2 to 4 days.
   Persistent or self-cleaning.—Self cleaning.
   Bud.—Shape: Narrowly conical Length: Approximately 1.0 cm Diameter: Approximately 0.5 cm Color: Bud base: RHS Green 137B with Yellow 4D. Apex flushed N6611 and N66C.
   Flower size.—Diameter: 3.5 cm. Height: 1.5 cm.
   Corolla/petals.—Arrangement: Single flower with 5 distinct petals fused at base. Length: Approximately 1.8 cm. Width: Approximately 1.5 cm. Quantity: 5 petals Texture: Glabrous Apex: Rounded, with shallow rounded lobes. Shape: Obovate Margin: Entire Color: When opening: Upper surface: RHS Red-Purple N74A Lower surface: RHS Red-Purple N74B Fully opened: Upper surface: RHS Red-Purple N74A, but much brighter Lower surface: RHS Red-Purple N74A and N74B.
   Calyx/sepals.—Quantity per flower: 2 Sepals per flower Shape: Deltoid, elliptic with pointed tip Length: 0.7 cm Width: 0.5 cm Apex: Acute Base: Fused Margin: Entire Texture: Glabrous all surfaces. Color: Upper Surface: RHS Yellow-Green 144A and Yellow 8D. Lower Surface: RHS Yellow-Green 144b and Yellow 8D.
   Peduncle.—Length: Approximately 0.5 cm. Diameter: Approximately 0.3 cm. Color: RHS Yellow-Green 144C Texture: Glabrous
   Fragrance.—No.
Reproductive organs:
   Stamens.—Number: Approximately 50 Filament length: Approximately 0.2 cm.
   Anthers.—Shape: Oval, bi-lobed Length: Less than 0.5 mm Color: RHS Yellow 8C Pollen: Color: Near RHS Yellow-Orange 23A Quantity: Moderate.
   Pistil.—Number: 1 per flower Length: 1.1 cm. Style: Length: 0.8 cm. Color: Near RHS Yellow 8C Stigma: Shape: 5 branched stigma Color: Near RHS Red-Purple 67A Ovary Color: Near RHS Yellow-Green 145D.
Other characteristics:
   Seeds and fruits.—Fruits are egg-shaped 4 mm long with a conical shape top. Seeds are dark grey to black, minute sphere, less than 1 mm. Too small to correctly identify RHS color.
   Disease/pest resistance.—Neither resistance nor susceptibility observed. Portulaca is not typically susceptible to many diseases and pests. The most common diseases being typical root rots from over watering, or black stem, Dichotomophthora portulaca. The most common pest of Portulaca is Aphid.
   Temperature tolerance.—Will not tolerate temperatures below 7° C. High temperature tolerance to at least 40° C.

What is claimed is:

1. A new and distinct cultivar of Portulaca plant named ‘PHSFR11-0’ as herein illustrated and described.
